(19) United States
(12) Patent Application Publication
(10) Pub. No.: US 2019/0234967 A1
(43) Pub. Date: Aug. 1, 2019

(54) Blood Test for Screening Out Amyloid and Alzheimers Disease Presence

(71) Applicant: University of North Texas Health Science Center at Fort Worth, Fort Worth, TX (US)

(72) Inventor: Sid E. O'Bryant, Aledo, TX (US)

(73) Assignee: University of North Texas Health Science Center at Fort Worth, Forth Worth, TX (US)

(21) Appl. No.: 16/312,346

(22) PCT Filed: Jun. 22, 2017
(86) PCT No.: PCT/US2017/038712

§ 371 Date: Dec. 21, 2018

Related U.S. Application Data
(60) Provisional application No. 62/353,360, filed on Jun. 22, 2016.

(51) Int. Cl. G01N 33/68

(52) U.S. Cl. CPC G01N 33/6896; G01N 2333/5409; G01N 2333/5412; G01N 2800/52; G01N 2333/7151; G01N 2800/2821; G01N 2333/5428

(57) ABSTRACT

The present invention includes a method for excluding patients from the need for further analysis of Alzheimer's Disease comprising: obtaining a blood or serum sample from a patient in a primary care setting; determining the expression levels of at least 4 of the following proteins: FABP, beta 2 microglobulin, PPY, soluble tumor necrosis factor receptor 1 (sTNFR1), CRP, VCAM-1, thrombopoietin, α2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1); comparing the level of expression from the sample with a statistically locked-down, multi-ethnic, broad age spectrum statistical sample; and determining if the patient is excluded from further testing for Alzheimer's Disease, thereby eliminating the need for further testing of the patient.

### STATEMENT OF FEDERALLY FUNDED RESEARCH

This invention was made with government support under AG039389 and AG12300 awarded by National Institutes of Health. The government has certain rights in the invention.

### TECHNICAL FIELD OF THE INVENTION

The present invention relates in general to the field of disease screening in primary care, specialty care or clinical trial setting, and more particularly, to a method of using a simple blood test to exclude patients from additional diagnostic procedures for Alzheimer's Disease, thereby reducing overall disease detection costs.

### BACKGROUND OF THE INVENTION

Without limiting the scope of the invention, its background is described in connection with blood marker screening.

Alzheimer's disease (AD) is the most common dementia and is the 5^{th} leading cause of death for those over 65 years of age¹. Currently, over 5 million Americans suffer from Alzheimer's disease (AD)². Furthermore, it is estimated that those numbers will grow exponentially by 2050. AD has an annual health care cost similar to that of cardiovascular disease (CVD) and more than cancer³. As a result of these rapidly increasing numbers, there is a growing need for the identification of a time- and cost-effective screening tool for use in primary care settings.

The Centers for Medicare and Medicaid Services recently implemented the Annual Wellness Visit (AWV) that includes a cognitive examination (CMS.gov); however, the 2015 American Gerontological Society working group reported that “older adults are inadequately assessed for cognitive impairment during routine visits with their primary care providers”⁴. This limited access to early diagnosis has been associated with delayed treatment initiation, delays in provision of services to family members, and an overall decreased quality of life and increased family burden⁵. Given the limited time available in primary care visits (average of 18 minutes), primary care providers are left with a significant dilemma of how to meet the AWV requirements.

### SUMMARY OF THE INVENTION

In one embodiment, the present invention includes a method for excluding patients from the need for further diagnostic procedures of Alzheimer's Disease comprising: obtaining a blood or serum sample from a patient in a primary care setting, specialty clinic setting or clinical trial setting; determining the expression levels of at least 4, 5, 6, 7, 8, 9, 10, 15, 20 or 21 of the following proteins: FABP, beta 2 microglobulin, PPY, soluble tumor necrosis factor receptor 1 (sTNFR1), CRP, VCAM-1, thrombopoietin, α2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1); comparing the level of expression from the sample with a statistically locked-down sample representative of the patient population, e.g., a multi-ethnic, broad age spectrum statistical sample; and determining if the patient is excluded from further diagnostic testing for Alzheimer's Disease from the comparison with the statistically locked-down, multi-ethnic, broad age spectrum statistical sample, thereby eliminating the need for further testing of the patient. In one aspect, the method further comprises the step of factoring the age, gender and education of the patient. In another aspect, the expression levels of 5, 6, 7, 8, 9, 10, 15, 20, or 21 of the proteins is determined. In another aspect, the method has a negative predictive value of greater than 0.95 for Alzheimer's Disease. In another aspect, the method has a positive predictive value of greater than 0.80 for Alzheimer's Disease. In another aspect, the method has a negative predictive value of greater than 0.90 for a mild cognitive impairment (MCI). In another aspect, the method has a positive predictive value of 0.4 or greater, e.g., 0.5, 0.6, 0.7, 0.8, for a mild cognitive impairment. In another aspect, the method has a negative predictive value of greater than 0.95 and a positive predictive value of 0.40 or greater, e.g., 0.5, 0.6, 0.7, 0.8, for Alzheimer's Disease. In another aspect, the method further comprises the step of avoiding additional diagnostic testing for Alzheimer's Disease wherein the diagnostic tests are selected from PET amyloid and/or tau scans, amyloid scanning methods, lumbar puncture for assay of amyloid, tau and other Alzheimer's diagnostic biomarkers, structural and functional MRI for ruling out Alzheimer's disease if the initial screen is negative for Alzheimer's Disease. In another aspect, the method further comprises the step of avoiding specific amyloid and/or tau-targeted additional treatments for Alzheimer's Disease wherein the treatment is selected from amyloid or tau diagnostic biomarkers (i.e. PET scan, lumbar puncture) if the initial screen is negative for Alzheimer's Disease. In another aspect, the screen comprises 5 protein markers and has a NPV of >=0.9 and PPV >=0.4 for AD, MCI and neurodegenerative disease. In another aspect, the screen comprises 5 protein markers and a cognitive test (e.g., electronic) to further improve on accuracy with a NPV>0.90. In another aspect, the screen comprises 5 protein markers and a cognitive test, an online or an electronic test, selected from at least one of clock drawing, verbal fluency, trail making test, MMSE or MoCA, and optionally further comprising determining an APOE4 genotype. In another aspect, the four proteins are IL10, IL5, IL6, and TNFα. In another aspect, at least three of the proteins are IL5, IL6 and TNFα.

Yet another embodiment of the present invention includes a method for excluding patients from the need for further diagnostic testing of Alzheimer's Disease comprising: obtaining a blood or serum sample from a patient in a primary care, specialty or clinical trial setting; determining the expression levels of at least 4, 5, 6, 7, 8, 9, 10, 15, 20 or 21 of the following proteins: FABP, beta 2 microglobulin, PPY, soluble tumor necrosis factor receptor 1 (sTNFR1), CRP, VCAM-1, thrombopoietin, α2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1); comparing the level of expression from the sample with a statistically locked-down sample representative of the patient population, e.g., a multi-ethnic, broad age spectrum statistical sample; and determining if the patient is excluded from further diagnostic testing for Alzheimer's Disease, thereby eliminating the need for further testing of the patient with a negative predictive value of greater than 0.90 for Alzheimer's Disease. In another aspect, the method further comprises the step of avoiding additional screening tests for Alzheimer's Disease wherein the screens are selected from PET amyloid and/or tau scans, amyloid scanning methods, lumbar puncture amyloid and/or tau procedures, structural MRI, and detailed neuropsychological testing if the initial screen is negative for Alzheimer's Disease. In another aspect, the method further comprises the step of avoiding additional treatments for Alzheimer's Disease wherein the treatment is selected from amyloid disease modifying therapies, tau therapies, cholinesterase inhibitors, NMDA receptor blockers and other Alzheimer's therapies if the initial screen is negative for Alzheimer's Disease. In another aspect, the screen comprises 5 protein markers and has a NPV or >=0.9 and PPV>=0.4 for AD, MCI and neurodegenerative disease. In another aspect, the screen comprises 5 protein markers and a select cognitive test (electronic) to further improve on accuracy with a NPV>0.90. In another aspect, the screen comprises 5 protein markers and a cognitive test, an online or an electronic test, selected from at least one of clock drawing, verbal fluency, trail making test, MMSE or MoCA, and optionally further comprising determining an APOE4 genotype. In another aspect, the four proteins are IL10, IL5, IL6, and TNFα. In another aspect, at least three of the proteins are IL5, IL6 and TNFα.

Yet another embodiment of the present invention includes a blood test adapted for use in a primary, specialty and clinical trial setting for excluding patients suspected of having Alzheimer's Disease comprising: one or more reagents that comprises a detectable marker adapted for use in a primary care setting, wherein the detectable marker is used to determine the expression levels of at least 4, 5, 6, 7, 8, 9, 10, 15, 20 or 21 of the following proteins: FABP, beta 2 microglobulin, PPY, soluble tumor necrosis factor receptor 1 (sTNFR1), CRP, VCAM-1, thrombopoietin, α2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1); a code segment that comprises an algorithm that determines the level of expression from the sample with a statistically locked-down sample representative of the patient population, e.g., a multi-ethnic, broad age spectrum statistical sample; and a processor that uses the code segment to determine if the patient is excluded from further diagnostic testing or treatment for Alzheimer's Disease, thereby eliminating the need for further testing of the patient with a negative predictive value of greater than 0.90 for Alzheimer's Disease.

In another aspect, the method further comprises a code segment for conducting a cognitive test to further improve on accuracy with a NPV>0.90. In another aspect, the screen comprises 5 protein markers (TNFα, CRP, IL7, ILS, IL6) and has a NPV or >=0.9 and PPV>=0.4 for AD, MCI and neurodegenerative disease. In another aspect, the screen comprises 5 protein markers (TNFα, CRP, IL7, ILS, IL6) and a select cognitive test (electronic) to further improve on accuracy with a NPV>0.90 and PPV>0.50. In another aspect, the screen comprises 5 protein markers and a cognitive test, an online or an electronic test, selected from at least one of clock drawing, verbal fluency, trail making test, MMSE or MoCA, and optionally further comprising determining an APOE4 genotype. In another aspect, the four proteins are IL10, ILS, IL6, and TNFα. In another aspect, at least three of the proteins are ILS, IL6 and TNFα.

In another embodiment, the present invention includes a method for excluding patients from recruitment into a clinical study by screening patients to rule out the presence of cerebral amyloid and/or tau comprising: obtaining a blood or serum sample from a patient; determining the expression levels of at least 4, 5, 6, 7, 8, 9, 10, 15, 20, or 21 of the following proteins: FABP, beta 2 microglobulin, PPY, soluble tumor necrosis factor receptor 1 (sTNFR1), CRP, VCAM-1, thrombopoietin, α2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1); comparing the level of expression from the sample with a statistically locked-down, multi-ethnic, broad age spectrum statistical sample; determining if the patient is unlikely to have cerebral amyloid and/or tau from the comparison with a statistically locked-down sample representative of the patient population, e.g., a multi-ethnic, broad age spectrum statistical sample; and excluding the patient from recruitment into the clinical study if the patient is ruled out of having the presence of cerebral amyloid and/or tau. In another aspect, the screen comprises 5 protein markers and a cognitive test, an online or an electronic test, selected from at least one of clock drawing, verbal fluency, trail making test, MMSE or MoCA, and optionally further comprising determining an APOE4 genotype. In another aspect, the four proteins are IL10, ILS, IL6, and TNFα. In another aspect, at least three of the proteins are IL5, IL6 and TNFα.

### BRIEF DESCRIPTION OF THE DRAWINGS

For a more complete understanding of the features and advantages of the present invention, reference is now made to the detailed description of the invention along with the accompanying figures and in which:

FIG. 1 shows a multi-state diagnostic process flowchart for detecting AD and discriminating AD from other dementias.

FIG. 2 shows a more detailed flowchart for detecting AD and discriminating AD from other dementias.

FIG. 3 shows the results of using the top 5 markers (TNFα, CRP, IL7, ILS, IL6) to detect AD, holding SP=0.95, SN fell to 0.50 which resulted in NPV=0.94 and PPV=0.53.

FIG. 4 is a graph that shows the sensitivity specificity for AD v NC serum using a 10 marker training set.

FIG. 5 is a graph that shows the sensitivity versus specificity for AD v NC serum using a 10 marker training set.

FIG. 6A and 6B show brain tissue sections from 3XTg (n=9) and control (n=9) mice [FIG. 6A] and human control (C n=9) and Alzheimer's disease (AD n=9) patients [FIG. 6B] were fixed and immunostained with primary antibodies to IL-6 or TNFα and fluorescence-labeled secondary antibody (green). The bar graphs on the right side of each of FIGS. 6A and 6B denote signal intensities of microvessels normalized to endothelial specific marker vonWillebrand factor (vWF, red) and control values set to 1. ***p<0.001.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention includes a blood test and method for excluding patients from the need for additional diagnostic testing that can fit into the current infrastructure and that is used to rule out patients who do not need further diagnostic workup. The present invention includes a novel blood-based screening tool for AD⁷⁻¹⁰ that can serve as first step in a multi-stage detection process¹¹ within community-based clinics, specialty clinics or clinical trial settings. Obtaining an early diagnosis within primary care settings can increase access to current therapies, reduce overall health care costs¹², delay nursing home placement¹³, facilitate a connection with community resources and reduce caregiver stress¹⁴ as well as assist in future planning¹⁵. This model follows the evolution of breast cancer screening in primary care¹⁶.

When designing a biomarker (blood-based or otherwise), it is crucial to define the context of use or fit-for-purpose¹⁷⁻¹⁹ and the desired performance of the biomarker itself. In this case, what is the overall purpose of the AD blood screener when applied to a primary care setting? Is it to “diagnose” AD or to determine who needs follow-up examination? In primary care settings (and other settings), a key context of use for nearly all screening tests is to rule out those who do not have the disease in order to decrease the numbers of patients that undergo more invasive and costly procedures. For example, a mammography does not rule-in breast cancer as the positive predictive value (PPV) is below 30%^{20,21}. Additionally, screening of depression in primary care have low PPVs (e.g., 0.15-0.27)²², but negative predictive power is excellent (>0.96)²². In both cases, the screening test ensures that only those who need the follow-up examination (biopsy, psychiatric referral) undergo such procedures, which serves as cost-containment and reduced unnecessary medical services to patients.

The present invention is a primary care AD blood screen that can be used to rule out 85% or more of elderly patients seen in primary care that do not need to undergo more expensive and invasive procedures. However, a screen positive on the AD blood test could trigger a multi-stage neurodiagnostic process of: (1) neurology specialty exam for differential purposes; (2) cognitive testing; and finally, (3) cerebrospinal fluid analysis and/or PET amyloid imaging. The AD blood screen lock-down referent sample consists of data from multiple clinic- and community-based settings and is multi-ethnic as required by fit-for-purpose biomarker validation methods. This AD blood screen yields excellent predictive power to determine which patients should NOT undergo additional expensive and invasive diagnostic methods, thereby offering a substantial cost savings to the health care system.

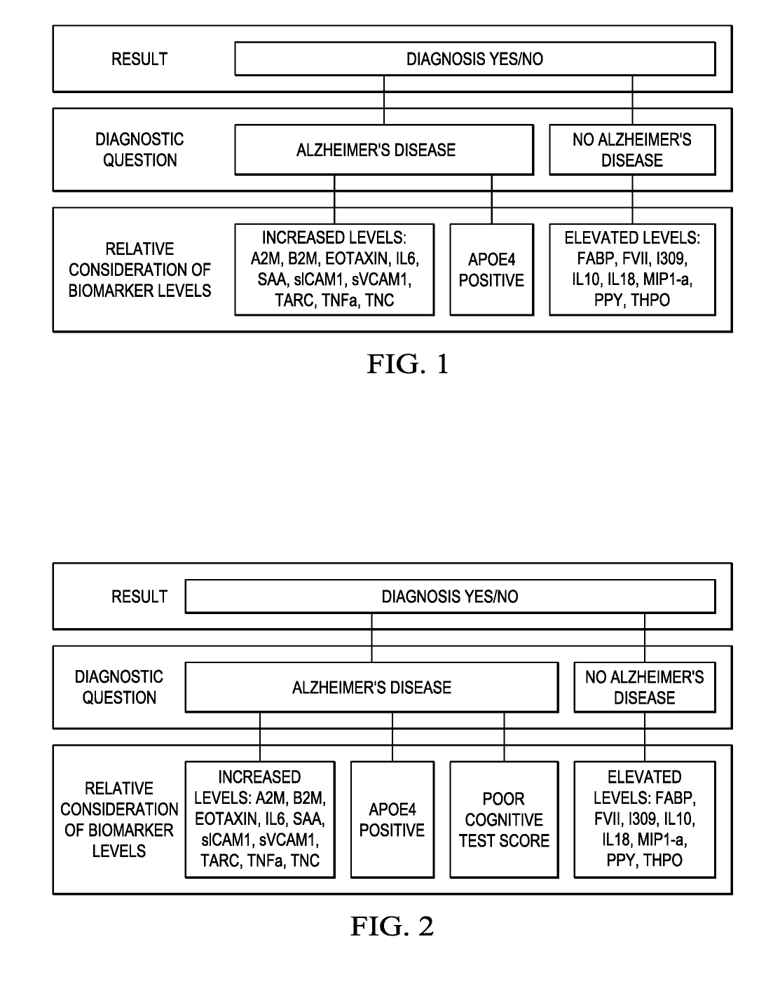

FIG. 1 provides an example of an updated patient flowchart for the multi-stage neurodiagnostic workup and differential diagnosis for AD. This process could be utilized for AD and non-AD dementias. In the first box, an initial diagnosis or result is to be determined to provide a yes or no answer based on the diagnostic question. In the middle box, the question is whether has Alzheimer's Disease been excluded. In the third box, a relative consideration is made about the expression levels of the various biomarkers listed, including increased levels of A2M, B₂M, Eotaxin, IL6, SAA, sICAM1, sVCAM1, TARC, TNFa, TNC, alternatively and in addition whether the patient is APO4 positive, and/or lastly if there are elevated levels of FABP, FVII, 1309, IL10, IL18, MIP1-a, PPY, THPO. In certain embodiments, 5 markers are selected that together provide a NPV of 0.8 or greater, e.g., 0.85, 0.90 or 0.95.

FIG. 2 provides an alternative flowchart shows, in the first box, an initial diagnosis or result is to be determined to provide a yes or no answer based on the diagnostic question. In the middle box, the question is whether has Alzheimer's Disease been excluded. The third box also includes increased levels of A2M, B₂M, Eotaxin, IL6, SAA, sICAM1, sVCAM1, TARC, TNFa, TNC, alternatively and in addition whether the patient is APO4 positive, and/or lastly if there are elevated levels of FABP, FVII, 1309, IL10, IL18, MIP1-a, PPY, THPO, but also includes the determination of a poor cognitive test score. The poor cognitive test score can be determined, at the primary care site using, e.g., a cognitive test, an online or an electronic test, selected from at least one of clock drawing, verbal fluency, trail making test, mini-mental state examination (MMSE) or Folstein test or Montreal Cognitive Assessment (MoCA), or equivalent thereof. In certain embodiments, 5 markers are selected that together with a cognitive test and/or APO4 genotyping provide a NPV of 0.8 or greater, e.g., 0.85, 0.90 or 0.95, and a PPV of 0.4 or greater.

Participants. Blood proteomic data was analyzed from 1,329 individuals across multiple community- and clinic-based cohorts.

Health & Aging Brain Among Latino Elders (HABLE)^{25,26}. Fasting samples were analyzed from the HABLE study, an ongoing epidemiological study of cognitive aging among community-dwelling Mexican Americans and non-Hispanic whites. The HABLE study utilizes a community-based participatory research (CBPR) approach, which involves partnering communities to conduct studies of human disease. This research was conducted under an IRB approved protocol with each participant (and/or informants for cognitively impaired persons) providing written informed consent. Each participant underwent an interview (i.e., medical history, medications, and health behaviors), detailed neuropsychological testing, blood draw, and medical examination (review of systems, Hachinski Ischemic Index scale, brief neurological screen). Testing was completed in English or Spanish depending on the participant's preference. Consensus diagnoses were assigned according to published criteria^{23,27}.

UTSW-Alzheimer's Disease Center. Samples from the NIA-funded UTSW ADC biorepository were analyzed. Each participant underwent an interview, neuropsychological testing, blood draw, and medical examination per the NACC protocol. Consensus diagnoses were assigned based on published criteria^{23,27-30}. Samples were drawn from clinic-based subjects and community-based subjects from prior ADC work.

Mayo Clinic, Jacksonville Alzheimer's Disease Center. Clinic-based samples were assayed from the NIA-funded Mayo Clinic Jacksonville ADC biorepository. Each participant underwent an interview, neuropsychological testing, blood draw, and medical examination per the NACC protocol. Consensus diagnoses were assigned based on published criteria^{23,27-30}.

Panama Aging Research Initiative (PARI) study³¹. Community-based samples were assayed from the PARI cohort, the first-ever study of Panamanian aging. PARI participants were recruited from the outpatient geriatric services of the largest public hospital of the Social Security (CSS) located in Panama, the capital of Panama. Each participant underwent an interview, cognitive testing and blood draw. All participants (or their proxies) signed informed consent forms and patient confidentiality was not breached in accordance with the Declaration of Helsinki (1964). Consensus diagnoses were assigned according to published criteria^{23,27}. Table 1 contains the demographic characteristics of each cohort.

TABLE 1
Demographic characteristics across cohorts

| | Total | | | HABLE | | | UTSW-ADC | | | PARI | | | MAYO | | |
|---|---|---|---|---|---|---|---|---|---|---|---|---|---|---|---|
| | AD | MCI | NC | AD | MCI | NC | AD | MCI | NC | AD | MCI | NC | AD | MCI | NC |
| Age | 75.8 | 69.4 | 65.8 | 74.2 | 66.3 | 59.2 | 72.6 | 69.0 | 67.6 | 82.3 | 81.7 | 76.9 | 76.8 | | 76.7 |
| | (8.6) | (8.6) | (9.8) | (9.0) | (8.0) | (6.7) | (8.1) | (7.2) | (7.3) | (9.1) | (7.8) | (6.7) | (5.7) | | (5.7) |
| | 51-103 | 50-102 | 50-95 | 58-91 | 50-86 | 50-85 | 51-103 | 52-94 | 50-84 | 66-97 | 69-102 | 65-95 | 64-88 | | 56-88 |
| Gender | 52.6 | 54.4 | 65.7 | 60.7 | 63.9 | 80.6 | 37.1 | 47.5 | 37.6 | 82.1 | 64.5 | 64.9 | 64.1 | | 72.9 |
| Female % | | | | | | | | | | | | | | | |
| Education | 13.1 | 11.4 | 11.8 | 5.6 | 6.9 | 8.8 | 14.6 | 14.6 | 15.5 | 6.8 | 6.8 | 9.2 | 13.7 | | 14.6 |
| | (4.3) | (5.2) | (4.9) | (4.6) | (4.6) | (4.6) | (2.8) | (2.9) | (2.4) | (3.4) | (2.9) | (3.9) | (3.0) | | (2.9) |
| | 0-22 | 0-22 | 0-23 | 0-16 | 0-20 | 0-20 | 8-20 | 7-22 | 10-22 | 0-14 | 1-12 | 1-16 | 6-20 | | 7-20 |
| Ethnicity | | | | | | | | | | | | | | | |
| % | | | | | | | | | | | | | | | |
| Non- | 61.8 | 41.7 | 31.9 | | 3.1 | 6.5 | 66.8 | 69.8 | 49.6 | | | | | | |
| Hispanic | | | | | | | | | | | | | | | |
| White | | | | | | | | | | | | | | | |
| Mexican | 14.4 | 41.7 | 49.5 | 100.0 | 96.9 | 93.3 | 0.5 | 1.7 | 0.4 | 100.0 | 100.0 | | 100.0 | | |
| American/ | | | | | | | | | | | | | | | |
| Latino | | | | | | | | | | | | | | | |
| African | 3.1 | 11.4 | 3.1 | | | | 6.8 | 19.6 | 11.9 | | | | | | |
| American | | | | | | | | | | | | | | | |
| Choctaw | 0.4 | 3.9 | 2.8 | | | | 1.0 | 6.7 | 10.6 | | | | | | |
| Other | | 1.3 | | | | | | 2.2 | | | | | | | |

Sample Collection. UTSW-ADC and PART samples were collected non-fasting while HABLE samples were collected fasting. Serum—(1) serum samples were collected into 10 mL tiger-top tubes; (2) samples were allowed to clot for 30 minutes at room temperature in a vertical position; (3) samples were centrifuged for 10 minutes at 1300×g at room temperature within one hour of collection; (4) 1.0 mL aliquots were transferred into cryovial tubes; and (5) samples were placed into −80° C. freezers for storage until use. Plasma—(1) blood was collected into 10 mL lavender-top (EDTA) tubes and gently inverted 10-12 times; (2) tubes were centrifuged at 1300×g at room temperature for 10 minutes within one hour of collection; (3) 1 mL aliquots were transferred to cryovial tubes; and (4) tubes were placed in −80° C. freezers for storage. Table 2 provides the breakdown of blood samples by diagnosis.

TABLE 2
Breakdown of final “locked down” referent cohort by diagnosis

| Diagnosis: | Sample Size |
|---|---|
| Normal Cognition | 613 |
| Parkinson's disease | 53 |
| Mild Cognitive Impairment | 309 |
| Alzheimer's Disease | 300 |
| Lewy Bodies Dementia | 53 |
| Vascular Dementia | 20 |
| Frontotemporal dementia | 19 |
| Total Sample | 1367 |

Proteomic Assays. Proteomic data was obtained in duplicate via a multiplex biomarker assay platform using electrochemiluminescence (ECL) on the SECTOR Imager 2400A from MSD (available at www.mesoscale.com). The MSD platform has been used extensively to assay biomarkers associated with a range of human diseases including AD. The markers assayed are from our previously validated AD blood screen and included: FABP, beta 2 microglobulin, PPY, soluble tumor necrosis factor receptor 1 (sTNFR1), CRP, VCAM-1, thrombopoietin, α2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1). Information regarding detectable limits (e.g. LDD) and other performance parameters of the assay platform (e.g. CVs, etc.) can be obtained from the first-author.

Statistical Analyses. Analyses were performed using IBM SPSS21 and R. Chi square and t-tests were used to compare case versus controls for categorical variables (sex, race) and continuous variables (age, education), respectively. Per the Institute of Medicine (TOM) guidelines³², a “locked down” referent cohort of n=1,128 individuals was created that was used for the referent in primary care settings and the remaining samples utilized for validation of the referent sample. All future clinical trials and other community-based projects looking at this AD blood screen will use this locked-down referent sample. This locked down cohort is multi-ethnic, community- and clinic-based and covers a broad age spectrum as is needed for implementation of a validated biomarker^{17,18}. Sensitivity, specificity and area under the receiver operating characteristic curve (AUC) were generated from the RF analyses. Positive predictive values (PPV) and negative predictive values (NPV) were calculated using a 12% estimated base rate of AD using Bayesian statistics³³.

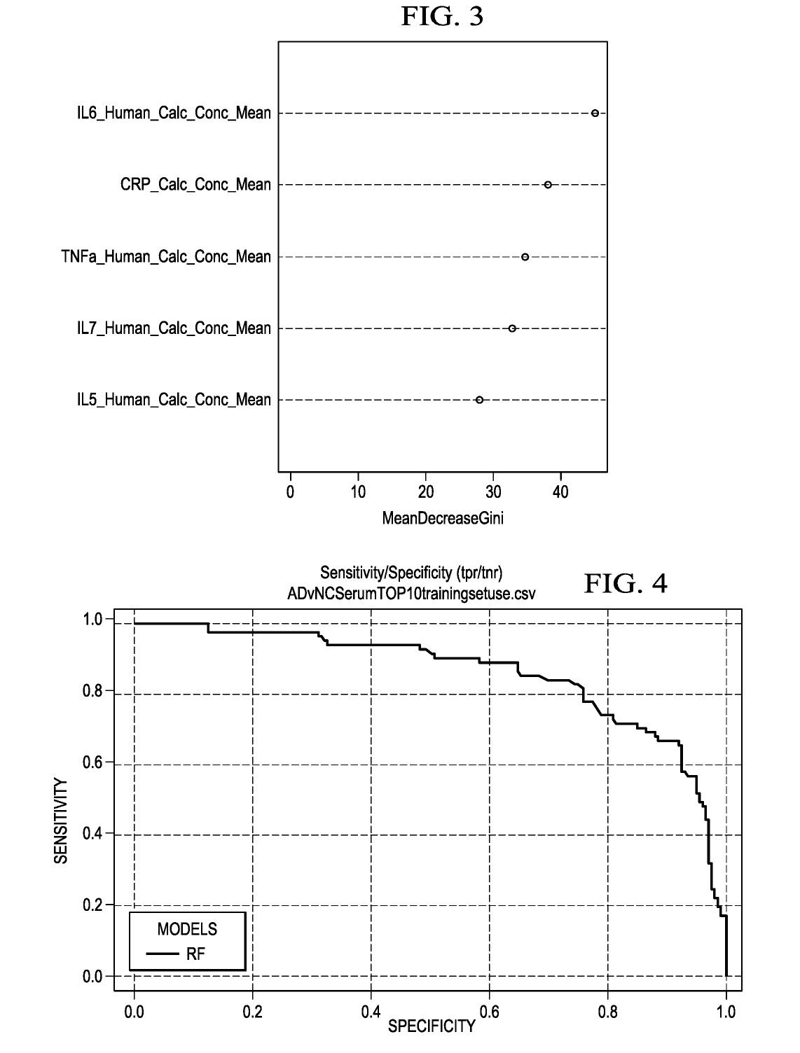

Table 1 provides the demographic characteristics of the sample. The referent “locked down” cohort (n=1,128; control n=613, AD n=255, MCI n=262) was utilized to detect AD among the remaining sample (n=201; control n=109, AD n=45, MCI n=47). For calculation of PPV and NPV, a population base-rate of 12% was used. Applying the primary care AD Blood Test from the “locked down” referent cohort, the 21-protein algorithm yielded an AUC was of 0.87. The addition of age, gender and education improved the AUC to 0.89. Therefore, PPV and NPV were calculated using the full algorithm of 21-proteins+demographics (age, gender and education). Setting specificity (SP) at 0.98, Sensitivity (SN) was 0.63 which resulted in a PPV of 0.81 and NPV=0.95. In an effort to consider cost reduction and scalability, the inventors restricted the AD Blood Test to only the top 10 proteomic markers (TNFa, 1309, sICAM1, CRP, IL10, TNC, FVII, IL6, IL7, IL5). The overall AUC was 0.90. When holding SP=0.98, SN fell to 0.58, which resulted in a PPV=0.80 and NPV=0.95. FIG. 3 shows the results of using the top 5 markers (TNFα, CRP, IL7, ILS, IL6) to detect AD, holding SP=0.95, SN fell to 0.50 which resulted in NPV=0.94 and PPV=0.53.

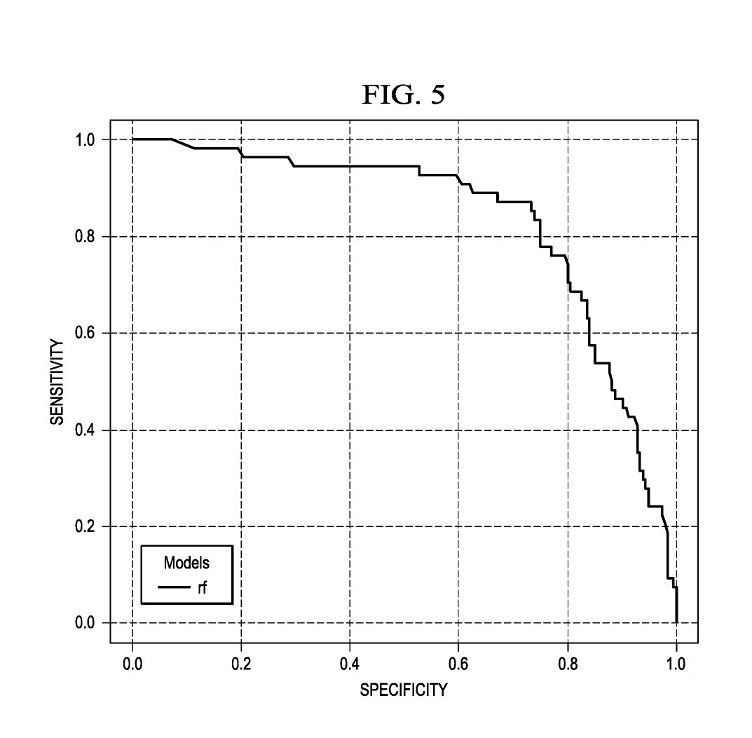

Next the referent “locked down” cohort was used to detect mild cognitive impairment (MCI). FIG. 4 is a graph that shows the sensitivity specificity for AD v NC serum using a 10 marker training set for the top 10 proteomic markers (TNFα, 1309, sICAM1, CRP, IL10, TNC, FVII, IL6, IL7, IL5). FIG. 5 is a graph that shows the sensitivity versus specificity for AD v NC serum using a 21 marker training set. Using the full 21-protein algorithm+demographics, the AUC was 0.88. With SP held at 0.98, SN was 0.42 which yielded a PPV=0.74 and NPV=0.93. When restricted only to the 10-protein+demographics algorithm, the AUC improved to 0.89. With SP set at 0.98, SN was 0.45, which resulted in a PPV=0.75 and NPV=0.93. Using the top 5 markers (TNFα, CRP, IL7, ILS, IL6) to detect MCI, holding SP=0.90, SN fell to 0.42, which resulted in a NPV=0.90 and PPV=0.43.

Preliminary analyses were also conducted to detect any neurodegenerative disease (Parkinson's Disease (PD), Lewy Body Dementia (LDB), Down Syndrome (DS), AD vs NC). Using the 21-protein+demographics AD blood test, the overall AUC was 0.92. Setting SP=0.98, SN was 0.62. Using a 15% base-rate of any neurodegenerative disease, PPV was 0.85 and NPV=0.94 for detecting any neurodegenerative disease (AD, PD, LDB, Frontotemporal dementia (FTD) and vascular dementia (VaD)). Using the top 10 markers, the AUC was 0.89. Holding SP=0.95, SN was 0.40 which resulted in a PPV=0.59 and NPV=0.90. Using the top 5 markers (TNFα, CRP, IL7, ILS, IL6) to detect any neurodegenerative disease, holding SP=0.95, SN fell to 0.40, which resulted in NPV=0.90 and PPV=0.59.

These results demonstrate that the AD blood test serves as a primary care tool to determine which patients warrant follow-up examination. As noted above, the purpose of this test is not diagnostic, but rather to provide a tool for assisting primary care physicians in making an empirically-based judgment on who requires a referral for more costly and invasive procedures. The availability of such a tool for primary care providers would serve to increase access to specialty clinics, CSF biomarker analysis and amyloid PET scans by reducing the numbers of inappropriate referrals.

The AD blood screen of the present invention provides an excellent NPV (0.95) and excellent PPV (0.80) for detecting AD. In fact, the AD blood test outperformed most screening instruments currently utilized in primary care. The AD blood screen was also excellent in ruling out MCI (NPV=0.93) and PPV was also very good (0.75). Given that the AD blood test was built for the context of use (COU) as a primary care screening tool for AD, this lower PPV is not surprising. However, when applied to MCI, the AD blood screen still performed comparable to or better than many commonly utilized primary care screens. Table 3 provides an overview of a broad range of screening tools for various conditions for comparison purposes.

TABLE 3
PPV and NPV of screening tests used in primary care
and other settings

| Screening Test | PPV | NPV | Context of use |
|---|---|---|---|
| Breast Cancer | | | |
| Mammography²¹ | .19-.29 | Not | Breast cancer screening in |
| | | provided | primary care |
| Digital | .12 | Not | Breast cancer screening in |
| mammography²⁰ | | provided | primary care |
| Geriatric Depression | | | |
| GDS-15²² | .15 | .99 | Depression screening in |
| | | | primary care |
| CES-D major dep²² | .27 | 1.0 | Depression screening in |
| | | | primary care |
| CES-D minor dep²² | .10 | .96 | Depression screening in |
| | | | primary care |
| Prostate Cancer | | | |
| PSA³⁸ | .06 | .97 | Prostate cancer screening |
| | | | and treatment monitoring |
| Gestational Diabetes | | | |
| Capillary blood | .20 | .95 | Screening for gestational |
| glucose³⁷ | | | diabetes |
| Hypertension | | | |
| Blood pressure | 0.35-0.95 | | OBPM screening for |
| testing³⁹ | | | hypertension as confirmed |
| | | | by ABPM or HBPM |
| Diabetic Ketoacidosis | | | |
| Urine dipstick³⁶ | 0.15 | 0.99 | Emergency department |
| | | | screening of DKA in |
| | | | hyperglycemic patients |
| B-OHB³⁶ | .35 | .99 | |
| Colorectal Cancer | | | |
| G-FOBT³⁵ | 0.35 | .99 | Colorectal Ca screening |
| | | | within hospital settings |
| FIT³⁵ | 0.11 | 0.99 | Colorectal Ca screening |
| | | | within hospital settings |
| CT colonography⁴⁰ | .62-.92 | .79-.97 | Follow-up colorectal cancer |
| | | | assessment with positive |
| | | | FOBT when colonoscopy |
| | | | is not available |
| HIV | | | |
| HIV screening ⁴¹ | 10.4 | .99 | Screening HIV in older |
| | | | children in primary care in |
| | | | high HIV prevalence |
| | | | settings |
| Lung Cancer | | | |
| Low Dose CT⁴² | .42 | .99 | Screening for lunch cancer |
| | | | with low-dose CT |

For example, the 15-item Geriatric Depression Scale yields a PPV=0.15 and NPV=0.99 for screening depression in a primary care setting²² when appropriate base rates are applied³⁴. The CES-D provided a PPV=0.27 and NPV=1.0 for major depression and PPV=0.10 and NPV=0.96 for minor depression²². Urine dipstick in an emergency room screening setting for detecting diabetic ketoacidosis (DKA) yields a PPV=0.15 but NPV=0.99. G-FOBT provides a PPV=0.35 and NPV=0.99 for detecting colorectal cancer³⁵. Low-dose CT for lung cancer screening provides a PPV=0.42 and NPV=0.99. PSA has a poor PPV, but excellent NPV³⁶. Capillary blood glucose only has a 20% PPV for detecting gestational diabetes but a NPV of 0.95³⁷.

As seen in Table 2, a host of screening instruments provide excellent NPV and therefore these initial screening tests screen out a tremendous number of patients who do not need subsequent examinations that are more invasive and costly. Therefore, our AD blood screen (and when applied to MCI) certainly performs within acceptable parameters when put within the primary care tool context of use and our final “locked down” sample containing all 1,367 samples is ready for clinical trial application (see Table 2). The present invention provides for the first time detection of neurodegenerative disease with the AD blood test at the primary care level that is also supportive of further examination.

In addition to serving as a method for primary care screening, the AD blood test also has a tremendous advantage for increasing access to disease modifying drugs (trials and medications when FDA approved). Specifically, application of the AD blood screen to rule out those who should not undergo PET amyloid imaging for inclusion into trials or consideration for treatment once FDA approval is acquired for one of these drugs. PET amyloid scanning is expensive and, as with cancer, not a viable first-line in determining drug intervention. If our AD blood screen provides a NPV=0.90 with a PPV=0.70 (lower than anticipated based on results above), this would reduce the PET amyloid scanning needs significantly. For example, using the MCI results above with SP=0.98 and SN=0.42, PPV=0.74 and NPV=0.93. If a total of 10,000 patients were screened for eligibility to PET scanning (for trial entry or drug administration), PET amyloid screening costs would be approximately $50M at $5,000 per scan (far less than the anticipated clinical cost of this scan). If the AD Blood Test is used as a first-step, it can accurately rule out 8,642 adults from receiving PET scans and reduce the PET scan screening cost by over $43 m. Again, a key purpose is to rule out those who do not need a PET scan. Availability of this AD blood screen can result in a significant cost savings of the screening budget for trials and a cost-savings when considering incorporating disease-modifying drugs into clinical practice. The FDA has yet to approve amyloid scanning methods, thus, the availability of this AD blood screen could also be used to achieve reimbursement for amyloid PET scans for those who screen positive on the blood test (i.e. cost-containment). Therefore, the availability of the AD blood test could also provide a cost-effective method for implementation of disease modifying drugs into the current medical system.

The AD blood screen of the present invention is a powerful tool for primary care physicians. This tool refines the diagnostic process such that those who screen positive undergo additional steps for the diagnosis as well as differential diagnosis. This process can also streamline and maximize cost-effectiveness of PET amyloid scans once disease-modifying drugs become FDA-approved.

Next, the inventors sought to determine if the same markers from the blood-based algorithm (above) were significantly altered in brain microvessels from Tg2576 AD mice and humans. Brain microvessels were fixed and immunostained with primary antibodies for IL6 and TNFa and fluorescence-labeled secondary antibody (green). The bar graph denotes signal intensities normalized to endothelial specific marker von Willebrand factor (vWF—red). Data are from 9 mice per group (p<0.001 vs. control). For humans, 100% of brain tissue sections from AD patients (n=9) and controls (n=9) were correctly identified.

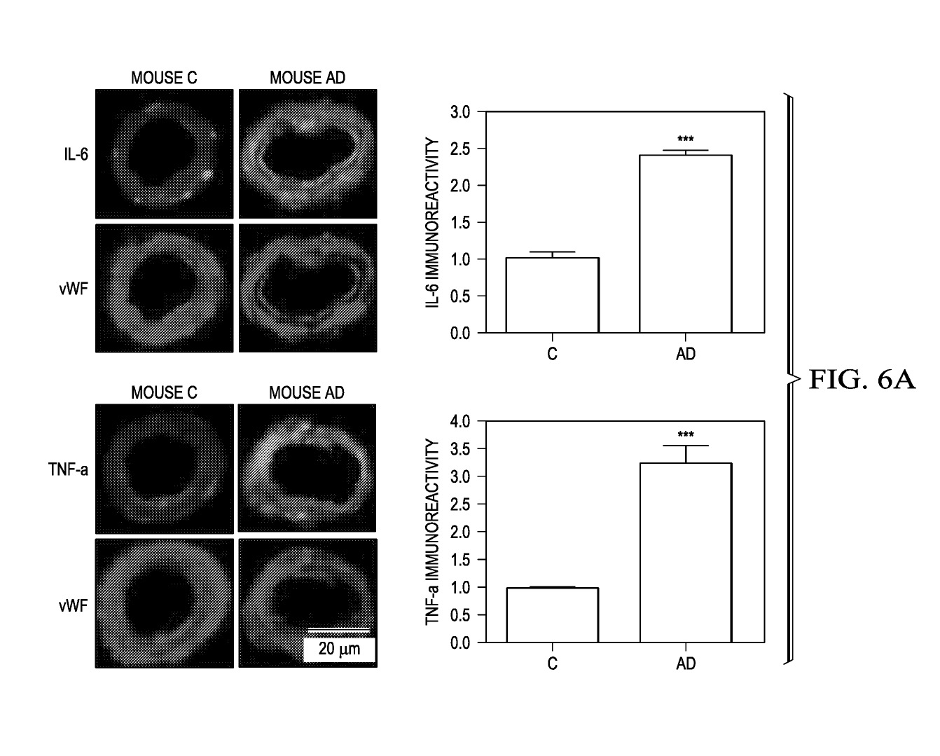

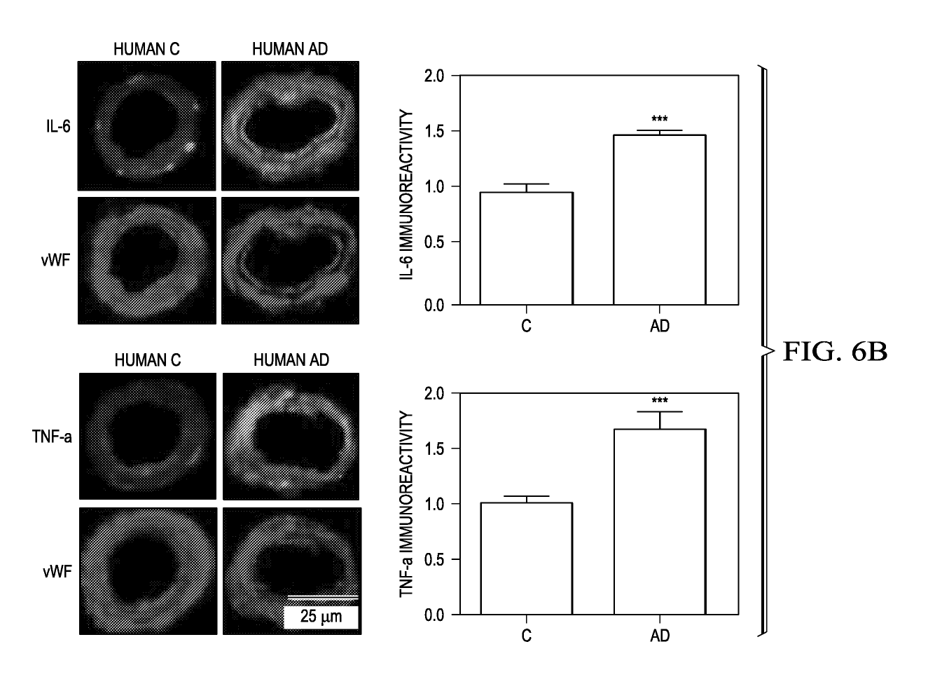

FIG. 6A and 6B show brain tissue sections from 3XTg (n=9) and control (n=9) mice [FIG. 6A] and human control (C n=9) and Alzheimer's disease (AD n=9) patients [FIG. 6B] were fixed and immunostained with primary antibodies to IL-6 or TNFα and fluorescence-labeled secondary antibody (green). The bar graphs on the right side of each of FIGS. 6A and 6B denote signal intensities of microvessels normalized to endothelial specific marker vonWillebrand factor (vWF, red) and control values set to 1. ***p<0.001.

Biomarkers for Alzheimer's disease should be cross-validated across human and animal models. Biomarkers were shown to be significantly altered in brain microvessels from 3XTg mice and human Alzheimer's disease patients. Furthermore, 100% of brain tissue sections from Alzheimer's disease patients (AD n=9) and human controls (C n=9) were correctly identified utilizing the present invention.

The present inventors further analyzed peripheral serum from 3XTg (n=9) and control (n=9) mice using ECL. The inventors assayed 4 of the top 8 markers (IL10, ILS, IL6, TNFα). Using logistic regression and four of the top biomarkers (IL10, ILS, IL6, TNFα), 99% of the mice were correctly classified. A 90% correlation was found with just three serum markers (IL5, IL6 and TNFα). Therefore, the present invention has been cross-validated across species using both brain tissue analysis and blood-based testing.

A study was conducted with Amyvid Aβ PET scans and blood biomarker analyses among 6 individuals (AD n=2, MCI n=2, control n=2). Four of the 6 participants were positive for Aβ (2 AD, 1 MCI and 1 control). The blood screen of the present invention was 100% accurate in detecting Aβ positivity. Again, the present invention was validated in humans using both brain tissue analysis and blood-based testing.

When put into the context of the Institute of Medicine (IOM) guidelines for steps from discovery to clinical utility, the AD Blood Test is the only work globally poised to undergo a full-scale clinical trial within the context of use of primary care settings. Such a trial is required for validation of the AD blood test. Additionally, this work establishes the “locked down” reference cohort for full-scale implementation of the methods and this referent cohort is the only globally available such cohort that covers clinic- and community-based adults and elders as well as multiple ethnicities.

Thus, the present invention provides for the first time an AD blood test for primary care settings that is cost- and time-effective for use in primary care settings and that makes a determination of which patients require follow-up examinations and procedures. The AD blood screen of the present invention also increases access to currently available medications and resources. Additionally, the availability of an AD primary care tool would increase access to more invasive diagnostic procedures (CSF or imaging biomarkers) as well as disease modifying drugs, once available. The AD blood screen performs equivalent to or better than many primary care screening examinations.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Alzheimer's Association. 2008 Alzheimer's disease facts and figures. Alzheimer's & Dementia. 2008;4(2):110-133.
2. Association As. 2013 Alzheimer's Disease Facts and Figures. Alzheimer's & Dementia. 2013;9(2):1-72.
3. Hurd M D, Martorell P, Delavande A, Mullen K J, Langa K M. Monetary Costs of Dementia in the United States. New England Journal of Medicine. 2013;368(14):1326-1334.
4. American GSo. The Gerontological Society of American Workgroup on Cognitive Impairment Detection: Report and Recommendations. 2015.
5. Novak K R, J. Hispanics/Latinos and Alzheimer's disease. Alzheimer's Association; May 18, 2004 2004.
6. O'Bryant S E, Xiao G, Zhang F, et al. Validation of a serum screen for alzheimer's disease across assay platforms, species, and tissues. Journal of Alzheimer's Disease. 2014;42(4):1325-1335.
7. O'Bryant S E, Xiao G, Barber R, et al. A serum protein-based algorithm for the detection of Alzheimer disease. Archives of Neurology. 2010;67(9):1077-1081.
8. O'Bryant S, Xiao, G, Barber, R, Reisch, J, Hall, J, Cullum, CM, Doody, R, Fairchild, T, Adams, P, Wilhelmsen, K, & Diaz-Arrastia, R. A blood based algorithm for the detection of Alzheimer's disease. Dementia and Geriatric Cognitive Disorders. 2011;32:55-62.
9. O'Bryant S E, Xiao G, Barber R, et al. A Blood-Based Screening Tool for Alzheimer's Disease That Spans Serum and Plasma: Findings from TARC and ADNI. PLoS ONE. 2011;6(12):e28092.
10. O'Bryant S E, Xiao G, Edwards M, et al. Biomarkers of Alzheimer's disease among Mexican Americans. Journal of Alzheimer's Disease. 2013;34(4):841-849.
11. Schneider P, Hampel H, Buerger K. Biological marker candidates of alzheimer's disease in blood, plasma, and serum. CNS Neuroscience and Therapeutics. 2009;15(4):358-374.
12. Fillit H, Hill J. Economics of dementia and pharmacoeconomics of dementia therapy. American Journal Geriatric Pharmacotherapy. 2005;3(1):39-49.
13. Mueller S G, Weiner M W, Thal L J, et al. Ways toward an early diagnosis in Alzheimer's disease: The Alzheimer's Disease Neuroimaging Initiative (ADNI). Alzheimer's and Dementia. 2005;1(1):55-66.
14. Connell C M, Roberts J S, McLaughlin S J, Carpenter B D. Black and white adult family members' attitudes toward a dementia diagnosis. Journal of the American Geriatrics Society. 2009;57(9): 1562-1568.
15. Knopman D, Donohue J A, Gutterman E M. Patterns of care in the early stages of Alzheimer's disease: Impediments to timely diagnosis. Journal of the American Geriatrics Society. 2000;48(3):300-304.
16. Lundquist T S, Ready R E. Screening for Alzheimer's disease: Inspiration and ideas from breast cancer strategies. Journal of Applied Gerontology. 2015;34(3):317-328.
17. Cummings J, Raynaud F, Jones L, Sugar R, Dive C. Fit-for-purpose biomarker method validation for application in clinical trials of anticancer drugs. British Journal of Cancer. 2010;103 (9): 1313-1317.
18. Jani D, Allinson J, Berisha F, et al. Recommendations for Use and Fit-for-Purpose Validation of Biomarker Multiplex Ligand Binding Assays in Drug Development. AAPS Journal. 2015.
19. Lee J W, Devanarayan V, Barrett Y C, et al. Fit-for-purpose method development and validation for successful biomarker measurement. Pharmaceutical Research. 2006;23(2):312-328.
20. Campari C, Rossi P G, Mori C A, et al. Impact of the Introduction of Digital Mammography in an Organized Screening Program on the Recall and Detection Rate. Journal of Digital Imaging. 2016;29(2):235-242.
21. Lee C S, Bhargavan-Chatfield M, Burnside E S, Nagy P, Sickles E A. The national mammography database: Preliminary data. American Journal of Roentgenology. 2016;206(4):883-890.
22. Watson L C, Pignone M P. Screening accuracy for late-life depression in primary care: A systematic review. Journal of Family Practice. 2003;52(12):956-964.
23. McKhann D, Drockman, D., Folstein, M. et al. Clinical diagnosis of Alzheimer's disease: Report of the NINCDS-ADRDA Work Group. Neurology. 1984;34:939-944.
24. Petersen R C, ed Mild Cognitive Impairment: Aging to Alzheimer's Disease. New York: Oxford University Press; 2003.
25. Szerlip H M E M, Williams B J, Johnson L A, Vintimilla R M & O'Bryant S E. Association of cognitive impairment with chronic kidney disease in Mexican Americans. Journal of the American Geriatric Society. 2015;63(10):2023-2028.
26. Johnson L A, Gamboa A, Vintimilla R, et al. Comorbid Depression and Diabetes as a Risk for Mild Cognitive Impairment and Alzheimer's Disease in Elderly Mexican Americans. Journal of Alzheimer's Disease. 2015;47(1):129-136.
27. Petersen R C, Negash S. Mild cognitive impairment: An overview. CNS Spectrums. 2008;13(1):45-53.
28. McKeith I G, Fairbairn A F, Perry R H, Thompson P. The clinical diagnosis and misdiagnosis of senile dementia of Lewy body type (SDLT). British Journal of Psychiatry. 1994;165(SEP.):324-332.
29. Anonymous. Clinical and neuropathological criteria for frontotemporal dementia. The Lund and Manchester Groups. Journal Of Neurology, Neurosurgery, And Psychiatry. 1994;57(4 (Print)): 416-418.
30. Emre M, Aarsland D, Brown R, et al. Clinical diagnostic criteria for dementia associated with Parkinson's disease. Movement Disorders. 2007;22(12):1689-1707.
31. Villarreal A E O B S, Edwards M, Grajales S & Britton G B for the Panama Aging Research Initiative. Serum-based protein profiles of Alzheimer's disease and mild cognitive impairment in elderly Hispanics. Neurodegener Dis Manag. 2016, in press.
32. Group B D W. Biomarkers and surrogate endpoints: preferred definitions and conceptual framework. Clin Pharmacol Ther.69:89-95.
33. O'Bryant S E, Lucas J A. Estimating the predictive value of the Test of Memory Malingering: An illustrative example for clinicians. Clinical Neuropsychologist. 2006;20(3):533-540.
34. Birrer R B, Vemuri S P. Depression in later life: A diagnostic and therapeutic challenge. American Family Physician. 2004;69(10):2375-2382.
35. Elsafi S H, Alqahtani N I, Zakary N Y, Al Zahrani E M. The sensitivity, specificity, predictive values, and likelihood ratios of fecal occult blood test for the detection of colorectal cancer in hospital settings. Clinical and Experimental Gastroenterology. 2015;8:279-284.
36. Arora S, Henderson S O, Long T, Menchine M. Diagnostic accuracy of point-of-care testing for diabetic ketoacidosis at emergency-department triage: O-hydroxybutyrate versus the urine dipstick. Diabetes Care. 2011;34(4):852-854.
37. Bhavadharini B, Mahalakshmi M M, Maheswari K, et al. Use of capillary blood glucose for screening for gestational diabetes mellitus in resource-constrained settings. Acta Diabetologica. 2016;53(1):91-97.
38. Harvey P, Basuita A, Endersby D, Curtis B, Iacovidou A, Walker M. A systematic review of the diagnostic accuracy of prostate specific antigen. BMC Urology. 2009;9(1).
39. Piper M A, Evans C V, Burda B U, Margolis K L, O'Connor E, Whitlock E P. Diagnostic and predictive accuracy of blood pressure screening methods with consideration of rescreening intervals: A systematic review for the U.S. Preventive Services Task Force. Annals of Internal Medicine. 2015;162(3):192-204.
40. Plumb A A, Halligan S, Pendsé D A, Taylor S A, Mallett S. Sensitivity and specificity of CT colonography for the detection of colonic neoplasia after positive faecal occult blood testing: Systematic review and meta-analysis. European Radiology. 2014;24(5):1049-1058.
   41. Bandason T, McHugh G, Dauya E, et al. Validation of a screening tool to identify older children living with HIV in primary care facilities in high HIV prevalence settings. AIDS. 2016;30(5):779-785.
   42. Sverzellati N, Silva M, Calareso G, et al. Low-dose computed tomography for lung cancer screening: comparison of performance between annual and biennial screen. European Radiology. 2016:1-9.

1. A method for excluding patients from the need for further analysis of Alzheimer's Disease comprising:
obtaining a blood or serum sample from a patient in a primary care setting;
determining the expression levels of at least four of the following proteins: FABP, beta 2 microglobulin, PPY, soluble tumor necrosis factor receptor 1 (sTNFR1), CRP, VCAM-1, thrombopoietin, α2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1);
comparing the level of expression from the sample with a statistical sample representative of the patient population; and
determining if the patient is excluded from further testing for Alzheimer's Disease from the comparison with the statistical sample, thereby eliminating the need for further testing of the patient.

2. The method of claim 1, wherein the statistical sample is a statistically locked-down, multi-ethnic, broad age spectrum statistical sample.

3. The method of claim 1, wherein the expression levels of 5, 6, 7, 8, 9, 10, 15, 20, or 21 of the proteins is determined.

4. The method of claim 1, further comprising the step of factoring the age, gender and education of the patient.

5. The method of claim 1, wherein the method has at least one of: a negative predictive value of greater than 0.95 for Alzheimer's Disease; a negative predictive value of greater than 0.90 for a mild cognitive impairment a positive predictive value of 0.4 or greater for Alzheimer's Disease; or a positive predictive value of 0.45 or greater for a mild cognitive impairment.

6. (canceled)

7. (canceled)

8. (canceled)

9. The method of claim 1, wherein the method has a negative predictive value of greater than 0.95 and a positive predictive value of greater than 0.80 for Alzheimer's Disease.

10. The method of claim 1, further comprising the step of avoiding additional screening tests for Alzheimer's Disease wherein the screens are selected from PET amyloid and/or tau scans, amyloid scanning methods, lumbar puncture amyloid and/or tau procedures, structural MRI, and detailed neuropsychological testing if the initial screen is negative for Alzheimer's Disease; or avoiding additional treatments for Alzheimer's Disease wherein the treatment is selected from amyloid disease modifying therapies, tau therapies, cholinesterase inhibitors, NMDA receptor blockers and other Alzheimer's therapies if the initial screen is negative for Alzheimer's Disease.

11. (canceled)

12. The method of claim 1, wherein the screen comprises at least one of: 5 protein markers are selected from TNFα, CRP, IL7, IL5, and IL6 that yield a NPV or >=0.9 and PPV >=0.4 for AD, MCI and neurodegenerative disease; 5 protein markers selected from TNFa, CRP, IL7, IL5, and IL6 and a select cognitive test to further improve on accuracy with a NPV>0.90; or 5 protein markers and a cognitive test, an online or an electronic test, selected from at least one of clock drawing, verbal fluency, trail making test, MMSE or MoCA, and optionally further comprising determining an APOE4 genotype .

13. (canceled)

14. (canceled)

15. (canceled)

16. The method of claim 1, wherein at least three of the proteins are IL5, IL6 and TNFα, or the four proteins are IL10, IL5, IL6, and TNFα.

17. A method for excluding patients from the need for further analysis of Alzheimer's Disease comprising:
obtaining a blood or serum sample from a patient in a primary care setting;
determining the expression levels of at least 4, 5, 6, 7, 8, 9, 10, 15, 20, or 21 of the following proteins: FABP, beta 2 microglobulin, PPY, soluble tumor necrosis factor receptor 1 (sTNFR1), CRP, VCAM-1, thrombopoietin, α2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1);
comparing the level of expression from the sample with a statistically locked-down, multi-ethnic, broad age spectrum statistical sample; and
determining if the patient is excluded from further diagnostic testing for Alzheimer's Disease, thereby eliminating the need for further testing of the patient with a negative predictive value of greater than 0.95 for Alzheimer's Disease.

18. The method of claim 17, further comprising the step of factoring the age, gender and education of the patient.

19. The method of claim 17, wherein the method has at least one of: a positive predictive value of 0.40 or greater for Alzheimer's Disease, a positive predictive value of 0.45 or greater for a mild cognitive impairment a negative predictive value of greater than 0.90 for a mild cognitive impairment or a negative predictive value of greater than 0.95 and a positive predictive value of greater than 0.80 for Alzheimer's Disease.

20. (canceled)

21. (canceled)

22. (canceled)

23. The method of claim 17, further comprising the step of avoiding additional screening tests for Alzheimer's Disease wherein the screens are selected from PET amyloid and/or tau scans, amyloid scanning methods, lumbar puncture amyloid and/or tau procedures, structural MRI and detailed neuropsychological testing if the initial screen is negative for Alzheimer's Disease.

24. The method of claim 17, further comprising the step of avoiding additional treatments for Alzheimer's Disease wherein the treatment is selected from amyloid disease modifying therapies, tau therapies, cholinesterase inhibitors, NMDA receptor blockers and other Alzheimer's therapies if the initial screen is negative for Alzheimer's Disease.

25. The method of claim 17, wherein the screen comprises at least one of: 5 protein markers that yield a NPV or >=0.9 and PPV>=0.4 for AD, MCI and neurodegenerative disease or 5 protein markers and a cognitive test to further improve on accuracy with a NPV>0.90, wherein the cognitive test is optionally computer based, or 5 protein markers selected are TNFα, CRP, IL7, IL5, and IL6 and a cognitive test, an online or an electronic test, selected from at least one of clock drawing, verbal fluency, trail making test, MMSE or MoCA, and optionally further comprising determining an APOE4 genotype.

26. (canceled)

27. (canceled)

28. (canceled)

29. The method of claim 17, wherein at least three of the proteins are IL5, IL6 and TNFα, or four proteins are IL10, IL5, IL6, and TNFα.

30. A blood test adapted for use in a primary care setting for excluding patients suspected of having Alzheimer's Disease comprising:
one or more reagents that comprises a detectable marker adapted for use in a primary care setting, wherein the detectable marker is used to determine the expression levels of at least 4, 5, 6, 7, 8, 9, 10, 15, 20, or 21 of the following proteins: FABP, beta 2 microglobulin, PPY, soluble tumor necrosis factor receptor 1 (sTNFR1), CRP, VCAM-1, thrombopoietin, α2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1);
a code segment that comprises an algorithm that determines the level of expression from the sample with a statistically locked-down, multi-ethnic, broad age spectrum statistical sample; and
a processor that uses the code segment to determine if the patient is excluded from further testing or treatment for Alzheimer's Disease, thereby eliminating the need for further testing of the patient with a negative predictive value of greater than 0.95 for Alzheimer's Disease.

31. The method of claim 30, further comprising a code segment for conducting a cognitive test to further improve on accuracy with a NPV>0.90.

32. (canceled)

33. The method of claim 30, wherein at least three of the proteins are IL5, IL6 and TNFα; or four proteins are IL10, ILS, IL6, and TNFα.

34. A method for excluding patients from recruitment into a clinical study by screening patients to rule out the presence of cerebral amyloid and/or tau comprising:
obtaining a blood or serum sample from a patient;
determining the expression levels of at least 4, 5, 6, 7, 8, 9, 10, 15, 20, or 21 of the following proteins: FABP, beta 2 microglobulin, PPY, soluble tumor necrosis factor receptor 1 (sTNFR1), CRP, VCAM-1, thrombopoietin, a2 macroglobulin, eotaxin 3, tumor necrosis factor-alpha (TNF-α), tenascin C (TNC), IL-5, IL-6, IL-7, IL-10, IL-18, 1309, Factor VII, thymus and activation-regulated chemokine (TARC), serum amyloid A (SAA), and intercellular cell-adhesion molecule-1 (ICAM-1), and optionally factoring the age, gender and education of the patient;
comparing the level of expression from the sample with a statistically locked-down, multi-ethnic, broad age spectrum statistical sample;
determining if the patient is unlikely to have cerebral amyloid and/or tau from the comparison with the statistically locked-down, multi-ethnic, broad age spectrum statistical sample; and
excluding the patient from recruitment into the clinical study if the patient is ruled out of having the presence of cerebral amyloid and/or tau; and optionally avoiding additional screening tests for cerebral amyloid and/or tau wherein the screens are selected from PET amyloid and/or tau scans, amyloid scanning methods, lumbar puncture amyloid and/or tau procedures, structural MRI, and detailed neuropsychological testing if the initial screening test rules out the presence of cerebral amyloid and/or tau.

35. (canceled)

36. (canceled)

37. (canceled)

38. (canceled)
